(19) United States
(12) Patent Application Publication
(10) Pub. No.: US 2009/0326035 A1
(43) Pub. Date: Dec. 31, 2009

(54) Compositions and Methods for Preventing or Reducing Postoperative Ileus and Gastric Stasis in Mammals

(76) Inventors: Uri Herzberg, Bridgewater, NJ (US); Scott Wadsworth, New Hope, PA (US); Kevin Cooper, Flemington, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/555,238

(22) Filed: Sep. 8, 2009

Related U.S. Application Data
(62) Division of application No. 11/761,707, filed on Jun. 12, 2007.
(60) Provisional application No. 60/813,250, filed on Jun. 13, 2006.

(51) Int. Cl. A61K 31/407; A61K 31/196; A61K 31/192

(52) U.S. Cl. 514/413; 514/567; 514/569

(57) ABSTRACT

Disclosed are compositions and methods for preventing or reducing postoperative ileus and gastric stasis. Such compositions include a combination of a carrier component and a bioactive component which acts to prevent or reduce post-operative ileus. Such methods include administering atherapeutically effective amount of the composition directly to the serosal surfaces of the gastrointestinal and other visceral organs.

### CROSS-REFERENCE TO RELATED APPLICATION

This application claims priority to U.S. provisional application Ser. No. 60/813,250 filed on Jun. 13, 2003 incorporated herein by reference in its entirety.

### FIELD

The present invention is directed to the prevention or reduction of postoperative ileus and gastric stasis in mammals, and compositions and methods of treatment.

### BACKGROUND

Postoperative ileus (also referred to herein as “POI”) and gastric stasis are problems that are common to most surgeries involving the abdomen. In addition, ileus and stasis are the main impediments for releasing a patient from the hospital.

Ileus is a major complication following abdominal surgery, and especially an abdominal surgery that involves manipulating the intestines and other abdominal organs. Specifically, paralytic ileus is the functional inhibition of peristaltic motility of the intestines. It prevents the absorption of drugs and nutrients, increases patient discomfort and pain, prolongs patient hospitalization and increases postoperative health care costs.

Care of the patient after surgery frequently does little to address the ileus condition and, in fact, often adds complications. Since opiates decrease intestinal motility, analgesic drugs such as morphine and codeine administered after surgery only exacerbate the severity and increase the incidence of postoperative ileus.

The main approaches for treating postoperative ileus and gastric stasis involve the use of systemic drugs. Currently there is only one drug, which has tentative approval for treating the condition. Specifically, a peripherally active opiate antagonist, Entereg (By Adolor and GlaxoSmithKline), has been submitted to the FDA for treating POI. Blocking peripheral opiate receptors is indeed a viable approach in preventing postoperative ileus. (See, for example, US 2002/0188005) Yet, this approach is only valuable in blocking the gastrointestinal effects of opiates which are commonly administered as analgesics following surgery. Such drugs are not likely to have an effect on the intestinal and gastric stasis that is opiates independent. Other approaches involve other mediators such as blocking the adenosine A1 receptor, blocking the Cox2 enzyme, and using the anti-inflammatory cytokine IL-11, etc. Blocking the PAR-2 enzyme has also been suggested as an approach (See, for example, WO 9843477.)

Common to all these approaches is the systemic delivery of an agent that enhances gastric and intestinal motility, or prevents the stasis and ileus of the stomach and intestines. However, systemic drug delivery, while simple, carries with it the side effect profile of the drug. For example, when systemically delivering anti-inflammatory drugs, such as NSAIDs, tissue healing will be affected. Cytokine injections such as IL-11 will affect the immune response of the patient, and systemic administration of A1 antagonists will have effects on the nervous system. Furthermore, for systemic drug delivery, non-target organs will also be affected.

While the pathology mediating the ileus and stasis condition is not clear, animal studies point to the activation of the cyclooxygenase enzymes (Cox1 and Cox2) as being at least partially responsible for this condition. While many Cox1 and Cox2 inhibitors are available, the systemic administration of Cox1 and Cox2 inhibitors to the post operative patient is, as with other systemically delivered agents, undesirable. This is due to their gastrointestinal side effect profile, their inhibitory effects on wound healing, their platelet inhibition and in the case of selective Cox2 inhibitors, such as Vioxx and Celebrex, their untoward cardiovascular side effect profile.

There is also evidence suggesting that mast cell degranulation and histamine release play a role in the induction and maintenance of POI (Demol et al. 1989, de Jonge et al. 2004). Following mast cell activation, histamine release initiates an inflammatory cascade whereby Cox1 and Cox2 enzymes are activated, nitric oxide synthase is activated and receptors for these mediators convey the information to neural centers. These neural centers reduce gastric emptying and block the coordinated motility of the intestines. Yet, the application of antihistamines, or mast cell degranulation inhibitors to the manipulated intestines in levels that are pharmacologically relevant to the problem without reaching systemic levels that will cause unwanted side effects remains a challenge. Given the foregoing considerations, there is a continuing need to identify treatment or prevention methods and compositions which are suitable and effective for preventing, treating and ameliorating postoperative ileus and gastric stasis. Such methods and compositions are ideally those which directly address the pathology of the condition but do not involve the systemic administration of drugs which can cause undesirable side effects.

### SUMMARY

Accordingly, the present invention provides compositions and methods for preventing or reducing gastric stasis and postoperative ileus, which compositions and methods involve direct administration of therapeutic agents to the serosal surfaces of the affected organs. The invention is directed to compositions suitable for administration to the serosal surfaces of the gastrointestinal and other visceral organs to prevent or reduce postoperative ileus and gastric stasis. Such compositions have a carrier component and a bioactive component which acts to prevent or reduce POI.

The invention is also directed to methods for preventing or reducing postoperative ileus and gastric stasis. Such methods provide for administering directly to the serosal surfaces of the gastrointestinal or other visceral organs an effective amount of a composition which comprises a carrier component and a bioactive component which acts to prevent or reduce POI.

These and other aspects and advantages of the present invention will be more apparent from the following description and accompanying drawings.

### BRIEF DESCRIPTION OF THE DRAWINGS

FIG. 1 is an elevation view of an applicator for applying a pharmaceutical agent effective in reducing POI

### DETAILED DESCRIPTION

To overcome the challenges of systemic administration of bioactive that reduces or prevents POI, the compositions of the presents invention locally deliver a bioactive to the affected site, such as the irritated serosal lining. By incorporating the bioactive component into the carrier component the underlying pathophysiology of POI can be addressed without the side effects and toxicity issues of systemic delivery.

Specifically, the drug is delivered to the area that has been manipulated by the surgeon in doses that are lower than the systemic daily doses required to achieve analgesia. By lowering the systemic doses, the side effects are also reduced.

The present invention is directed to compositions and methods for preventing or reducing POI by direct administration of a composition comprising a carrier component and a bioactive component which acts to prevent or reduce POI to the serosal surfaces of the gastrointestinal and other visceral organs.

### Carrier Component

The carrier component of the compositions of the present invention is used to locally deliver the bioactive component to the site of the surgery. The carrier component can be in a number of physical forms including, but not limited to injectable or sprayable gels or liquids. The bioactive component is incorporated into the carrier component. Using injectable or sprayable gels or liquids that can deliver such drugs locally will have the beneficial effect of reducing the incidence and severity of ileus, while not reaching plasma levels that will induce side effects such as sedation, or interfering with other systemic drugs that the patient is on.

The carrier component may be an injectable or sprayable gel or liquid. The injectable or sprayable gel or liquid is comprised of an aqueous solvent and a gelling material. Suitable aqueous solvents include, but are not limited to physiological buffer solution, saline and water such as, buffered saline, phosphate buffer solution, Hank's balanced salts solution, Tris buffered saline, and Hepes buffered saline. In one embodiment, the aqueous solvent is phosphate buffer solution.

Suitable gelling materials include, but are not limited to proteins such as, collagen, elastin, thrombin, fibronectin, gelatin, fibrin, tropoelastin, polypeptides, laminin, proteoglycans, fibrin glue, fibrin clot, platelet rich plasma (PRP) clot, platelet poor plasma (PPP) clot, self-assembling peptide hydrogels, and atelocollagen; polysaccharides such as, starch, pectin, cellulose, alkyl cellulose, e.g. methylcellulose, alkylhydroxyalkyl cellulose, hydroxyalkyl cellulose, cellulose sulfate, salts of carboxymethyl cellulose, carboxymethyl cellulose, carboxyethyl cellulose, chitin, carboxymethyl chitin, hyaluronic acid, salts of hyaluronic acid, alginate, cross-linked alginate alginic acid, propylene glycol alginate, glycogen, dextran, dextran sulfate, curdlan, pectin, pullulan, xanthan, chondroitin, chondroitin sulfates, carboxymethyl dextran, carboxymethyl chitosan, chitosan, heparin, heparin sulfate, heparan, heparan sulfate, dermatan sulfate, keratan sulfate, carrageenans, chitosan, starch, amylose, amylopectin, poly-N-glucosamine, polymannuronic acid, polyglucuronic acid polyguluronicacid, and derivatives; polynucleotides such as, ribonucleic acids, deoxyribonucleic acids, and others such as, poly(N-isopropylacrylamide), poly(oxyalkylene), copolymers of poly(ethylene oxide)-poly(propylene oxide), poly(vinyl alcohol), polyacrylate, monostearoyl glycerol co-succinate/polyethylene glycol (MGSA/PEG) copolymers and combinations thereof.

In one embodiment, the gelling material includes, but is not limited to polysaccharides. In another embodiment, the gelling material is sodium carboxymethylcellulose.

The injectable or sprayable gel or liquid may be prepared by dissolving an effective amount of gelling material in the aqueous solvent. An “effective amount” of gelling material is defined as the amount of gelling material sufficiently necessary to allow the injectable or sprayable gel or liquid to be either injected into or sprayed onto the affected area and stay in place upon application. The effective amount of gelling material will vary depending upon the material chosen. One of skill in the art may easily determine an effective amount of gelling material for the desired material. In one embodiment, where the gelling material is sodium carboxymethylcellulose the gelling material is present in an amount of about 0.1 weight % to about 5 weight % in the aqueous solvent. In another embodiment, the gelling material is present in an amount of about 0.5 weight % to about 3 weight % in the aqueous solvent. The injectable or sprayable gel or liquid may be in a gel form prior to injection or may be in a liquid form and gel and stay in place upon administration.

### Bioactive Component

The compositions and methods of the present invention involve a carrier component used to locally deliver a bioactive component to the site of the surgery. The bioactive components are preferably non-steroidal, anti-inflammatory drugs (NSAIDs). In one embodiment, the bioactive component functions as an inhibitor of the activity of cyclooxygenase (Cox1 and/or Cox2) enzymes referred to herein as NSAID Cox inhibitors. The NSAID Cox inhibitors can be those which are either non-selective or selective. Non-selective Cox inhibitors are those which inhibit the activity of both the Cox1 and Cox2 variants of the cyclooxygenase enzyme. Selective Cox inhibitors are those which selectively inhibit the activity of either the Cox1 or Cox2 enzyme forms preferentially.

Suitable NSAID Cox inhibitors include, but are not limited to propionic acid derivatives; acetic acid derivatives; fenamic acid derivatives; biphenylcarboxylic acid derivatives; and combinations thereof. These acids are sometimes administered in the form of their pharmaceutically acceptable acid or in the form of their alkali metal salts, e.g., sodium salts.

Propionic acid derivatives include, but are not limited to ibuprofen, naproxen, ketorolac, benoxaprofen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen bucloxic acid and the like. Structurally related propionic acid derivatives having similar cyclooxygenase inhibiting properties are also intended to be encompassed by this group.

Acetic acid derivatives include, but are not limited to indomethacin, sulindac, tolmetin, zomepirac, diclofenac, bromofenac, fenclofenac, alclofenac, ibufenac, isoxepac, furofenac, tiopinac, zidometacin, acemetacin, fentiazac, clidanac oxpinac, and the like. Structurally related acetic acid derivatives having similar cyclooxygenase inhibiting properties are also intended to be encompassed by this group.

Fenamic acid derivatives include, but are not limited to mefenamic acid, meclofenamic acid, flufenamic acid, niflumic acid tolfenamic acid and the like. Structurally related fenamic acid derivatives having similar cyclooxygenase inhibiting properties are also intended to be encompassed by this group.

Biphenylcarboxylic acid derivatives include, but are not limited to diflunisal, flufenisal, and the like. Structurally related biphenylcarboxylic acid derivatives having similar cyclooxygenase inhibiting properties are also intended to be encompassed by this group.

In one embodiment, the NSAID Cox inhibitors include, but are not limited to ibuprofen, naproxen, flurbiprofen, fenoprofen, ketoprofen, fenbufen, zomepirac, diclofenac, ketorolac, bromofenac, indomethacin, mefenamic acid, meclofenamate acid, diflunisal, flufenisal, sodium salts thereof, and combinations thereof.

In another embodiment, the NSAID Cox inhibitors are acids having a secondary amine group. NSAID Cox inhibitors that are acids having a secondary amine group include, but are not limited to diclofenac; diclofenac, monosodium salt; mefenamic acid, monosodium salt; and bromofenac (bromide salt of diclofenac).

Cox inhibitors of all of the foregoing types, as well as other NSAIDs suitable as Cox inhibitors, are described in greater detail in U.S. Pat. Nos. 6,689,382 and 6,231,888, United States Patent Publication No. 2003/0212050 and European Patent Application No. EP-A-485,111 incorporated herein by reference in their entirety.

It is understood that the present invention contemplates the use of not only the NSAID Cox inhibitor compounds themselves, but also their pro-drugs which metabolize to the compounds and the analogs and biologically active salt forms thereof, as well as optical isomers which provide the same pharmaceutical results. Thus for purposes of this invention, the term “non-steroidal anti-inflammatory drug” is meant to include all derivatives or precursors such as salts, esters, pro-drugs, analogs, isomers, etc. which are NSAIDs themselves or which can yield materials which function as NSAIDs.

### Composition Preparation Procedures and Preparation Adjuvants

The carrier component may be an injectable or sprayable gel or liquid. Also, it may be appropriate to utilize various types of adjuvants in preparing such compositions.

The carrier component is used to locally deliver the bioactive component to the site of the surgery. The bioactive component is incorporated into the carrier component. The bioactive component may be combined with the carrier component by manually mixing the components together or by conventional mechanical mixing such as, a motor driven rotating mixing paddle or blade. The mixing may be accomplished at ambient temperature. Using an injectable or sprayable gel or liquid that can deliver such drugs locally has the beneficial effect of reducing the incidence and severity of ileus, while not reaching plasma levels that will induce side effects such as sedation, or interfering with other systemic drugs that the patient is on.

The therapeutically effective concentration of a bioactive component will be one that is sufficient such that when delivered locally to the irritated or otherwise manipulated organ it will prevent or decrease the severity of post operative ileus. The maximum amount of the bioactive component will be limited by the toxicity of the bioactive component either at the local tissue or due to systemic levels.

The mass ratio of the bioactive component to the carrier component can typically range from about 1:50,000 to about 1:100. In one embodiment, this mass ratio of bioactive component to carrier component will range from about 1:30,000 to about 1:1,000.

The mechanism of release of the bioactive component from the carrier component should be in accordance with the development and maintenance of postoperative ileus. Hence, a release over 3-5 days will be preferable. Release over a shorter period of time is also acceptable, provided that the bioactive components prevent or reduce the development of ileus following the surgical intervention.

The compositions may also benefit from having a variety of optional substances included such as stabilizers, wetting agents, or preservatives. Other drugs may also be added to the composition, so long as it is compatible with the bioactive component and the remaining ingredients. These drugs include antibiotics, antiviral, and anti-fungal agents. Since tissue sites are occasionally infected, an antibiotic or anti-microbial agent may also be combined with the composition.

### Use of Composition to Prevent or Reduce Postoperative Ileus and Gastric Stasis

The methods of the present invention provide for administering directly to the serosal surfaces of the gastrointestinal or other visceral organs a therapeutically effective amount of a composition of the present invention as hereinbefore described. Such a composition utilizes a carrier component for local delivery of a bioactive component which acts to prevent or reduce POI. Compositions, as described herein, are useful in variety of abdominal, uro-gynecological, and cardio-thoracic surgeries. The composition may be applied after manipulation of serosal surfaces of the gastrointestinal or other visceral organs prior to closure to reduce or prevent POI. The composition may be applied by injecting or spraying the composition onto the serosal surfaces of the gastrointestinal or other visceral organs.

The compositions of the present invention containing a pharmaceutical agent effective in reducing POI may be formed into a gel, slurry, bead, sponge, or the like. A gel, slurry, bead, or sponge containing a pharmaceutical agent effective in reducing POI may be applied to the interior or the exterior of the intestine in a manner consistent with those described for powders by using an instrument that can pump or push the composition to the desired location within the patient. U.S. Pat. No. 6,251,063 teaches a delivery method and gun or syringe that can be used to introduce the gel, slurry, bead, sponge, or the like. The needle tip of the gun or syringe may be fitted with a wider blunt tip as needed to apply the varying thickness and consistencies for the gel, slurry, bead, sponge, or the like. Additionally, using the method taught in U.S. Pat. No. 6,251,063 the compositions may be injected into the wall of the intestine to reduce POI.

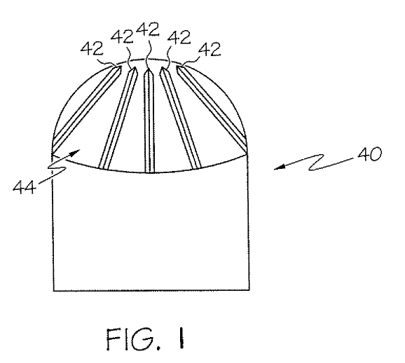

FIG. 1 shows a roll-on applicator 40 that may be filled with a gel or slurry composition containing a pharmaceutical agent effective in reducing POI. Using the roll-on applicator 40 the NSAID may be applied to the desired location of the intestine to reduce POI. The roll-on applicator 40 could be smooth, or in one embodiment be provided with multiple channels or groves, shown as v-shaped grooves 42 in the surface of the applicator tip 44. The grooves or channels 42 need not be v-shaped, but may assume any desired shape so long as the function of channeling the gel or slurry composition containing a NSAID to the application area is achieved. For example, the grooves or channels 42 may be v-shaped, u-shaped, square, semi-circular, semi-oval, or any other geometric shape. The grooves or channels 42 may be formed such that they are exposed on the surface, or they may be formed to be located beneath the surface of the applicator tip 44. Additionally, the applicator tip 44 need not be dome-shaped, as shown in FIG. 1, but may be any suitable applicator tip shape that applies the gel or slurry composition containing a NSAID to the area to be treated for POI or gastric stasis.

The roll-on applicator may be used in laparoscopic surgery, open surgery, or the like. In another embodiment a laparoscopic device with a rigid or flexible shaft of varying length may be fitted with the roll-on applicator. The laparoscopic device may be used to reach into the patient's body to apply the NSAID at the desired location.

The compositions of the present invention effective in reducing POI may be packaged as shown in U.S. Pat. No. 6,372,313. In one embodiment, the kit may contain the NSAID composition in a container, bottle, ampoule, pouch, sealed individual depressions, or the like of various sizes. The kit may also contain appliers of various sizes and shapes. The NSAID composition may be a solution capable of being placed onto an applier drop by drop. The NSAID may be a solution, slurry, or gel capable of having the applier dipped into the container and extracted containing a small amount of the NSAID ready for application to the patient's intestine or abdominal cavity. The NSAID may be a solid, powder, slurry, gel, solution, or the like stored in an ampoule that comes with an applier as shown in U.S. Pat. No. 6,340,097. The ampoule may have a shield that separates the NSAID composition from the air and must be broken by the applier to reach the composition.

The NSAID composition may need to be sterilized before use in treating POI. The NSAID composition may be packaged according to the methods in U.S. Pat. No. 6,412,639. The NSAID composition can be sterilized separately from the surgical tools and then remain sterile by separating the NSAID composition from the sterilization used for the surgical tools by way of a sterilization barrier built into the kit.

The following examples are illustrative of the principles and practice of the present invention, although not limited thereto.

### EXAMPLES

To illustrate the present invention, several exemplary compositions containing a carrier component and bioactive components were prepared. These compositions were also evaluated via in vivo testing for their ability to alleviate postoperative ileus.

### Example 1

### Preparation of Diclofenac Sodium Loaded Gel

Sodium carboxymethylcellulose (Na-CMC, Type 7H3SFPH, Hercules, Willmington, Del.) gels were loaded with Diclofenac sodium (DFNa, cat. #D6899-100G, Sigma-Aldrich, St. Louis, Mo.) using a physical mixing. Two drug loadings (high dosage and low dosage) were prepared. Ten grams of Na-CMC were slowly added into 290 grams of phosphate buffered saline (PBS) solution with magnetic stirring at 60° C. to prepare 3 wt % Na-CMC solution. The pre-mixed solution was autoclaved using AMSCO Steam Powered Sterilizer (Model #3021, SN 0100593-08) at 121° C. for 20 minutes to make a homogenous gel solution. The high dosage gel composition (1 mg DFNa/3 g Na-CMC), was prepared by adding 13 mg of DFNa to 39 grams of 3 wt % Na-CMC gel. The mixture was well mixed manually with a spatula for 5 minutes. 3 grams of DFNa high dose composition were loaded into 5 cc disposable syringes. The low dosage gel composition (0.3 mg DFNa/3 g Na-CMC), was prepared by adding 3.9 mg of DFNa to 39 grams of 3 wt % Na-CMC gel. The mixture was well mixed by hands for 5 minutes. 3 grams of DFNa low dose composition were loaded into 5 cc disposable syringes. 3 grams of 3 wt % Na-CMC gel were loaded into 5 cc disposable syringes for the gel only treatment.

### In Vivo Testing of the Effects of Diclofenac Gel on POI

The charcoal transit model is an acceptable and established model to study the effects of abdominal surgery on intestinal motility. We followed the procedure as described by De Winter et al. (1997) (Reference: Effect of adrenergic and nitrergic blockade on experimental ileus in rats. British Journal of Pharmacology (1997) 120, 464-468. Benedicte Y. De Winter, Guy E. Boeckxstaens, Joris G. De Man, Tom G. Moreels, Arnold G. Herman & Paul A. Pelckmans). The model simulates the abdominal procedure in human and tests for gastric emptying and intestinal motility by the transfer of a charcoal meal fed to the animals.

### Methods of Treatment

### Surgeries

Male Sprague Dawley rats, weighing 250-275 g (8 in group) were anesthetized with a xylazine/ketamine cocktail, injected IM, the abdomen was shaved and disinfected with alcohol and Betadyne. A 2.5 cm incision was made from the xyphoid process caudally. The intestinal contents was exteriorized onto wet gauze. The cecum was manipulated gently for 1 minute. The abdominal contents were replaced, the treatment was applied, and the body wall was closed with 4-0 PDS for the fascia and muscle layers. The skin was closed with wound clips. In addition, two control groups were tested as well: incision only and naïve. Treatment groups included two control groups, surgery (incision only) and naïve, and two treatment groups, a high and low dose of Diclofenac, as well as 3 wt % Na-CMC gel alone. The high and low dose of Diclofenac, as well as the 3 wt % Na-CMC gel were prepared as described above. 8 rats were included in each group with a total of 40 rats.

### Charcoal Transit

The rats were treated with an oral solution of charcoal (10%) in acacia gum (2%). Four hours following oral administration rats were euthanized with Euthasole. The intestines were exteriorized and the distance traveled by the charcoal meal was measured as well as the full length of the intestines (from the pylorus through the anus). Results are expressed as percent of distance traveled relative to the full length of the intestines.

### Study Groups

Gel composition

| | | Total dose |
|---|---|---|
| Drug | Dose (mg/ml) | (mg) |
| Gel only | 0.00 | 0 |
| Diclofenac-Na | 0.10 | 0.3 |
| low | | |
| Diclofenac-Na | 0.33 | 1 |
| high | | |

### Results

| | Dose | % GI | |
|---|---|---|---|
| Study Group | (mg) | Transfer | SE |
| Naïve (no surgery) | NA | 92.63 | 0.76 |
| Control (surgery - no | NA | 37.79 | 1.01 |
| treatment) | | | |
| Gel only | NA | 38.76 | 1.81 |
| Diclofenac-Na low | 1.0 | 95.05 | 1.23 |
| Diclofenac-Na high | 0.3 | 73.58 | 0.90 |

It was observed that intestinal manipulation decreases gastric emptying and intestinal motility in the rat in the same manner that has been described in humans undergoing intestinal surgery. Diclofenac sodium, when delivered via a gel, to the manipulated intestines, prevents the decrease in gastric emptying and GI motility in a dose related manner. A complete prevention was observed when the high dose was used and a significant prevention of POI was observed when the low dose was used. The gel alone had no significant effect on the POI.

### Example 2

### Naproxen Loaded Gel

Na-CMC gels loaded with Naproxen (lot #: 076K15861 Sigma-Aldrich, St. Louis, Mo.) were prepared using physical mixing. Two loadings (high dosage and low dosage) were prepared. 10 grams of Na-CMC were slowly added into 290 gram of PBS solution with magnetic stirring at 60° C. to prepare 3 wt % Na-CMC solution. The pre-mixed solution was autoclaved at 121° C. for 20 minutes to make a homogenous gel solution.

The high dosage gel composition (2 mg Naproxen/3 g Na-CMC) was prepared by adding 24 mg of Naproxen to 36 g of 3 wt % Na-CMC gel. The mixture was manually mixed with a spatula for 5 minutes. 3 g of gel were weighed into 5 ml syringes with a 3 way stopcock. Syringes were autoclaved for 20 minutes at 121° C. using an AMSCO Steam Powered Sterilizer (Model #3021, SN 0100593-08).

The low dosage gel composition (0.3 mg Naproxen/3 g Na-CMC) was prepared by adding 3.6 mg of Naproxen to 36 g of 3 wt % Na-CMC gel. The mixture was manually mixed using a spatula for 5 minutes. 3 g of gel were weighed into 5 ml syringes with a 3 way stopcock. Syringes were autoclaved for 20 minutes at 121° C. using an AMSCO Steam Powered Sterilizer (Model #3021, SN 0100593-08).

### Ketorolac Loaded Gel

Na-CMC gels loaded with Ketorolac (lot#:083K0734, Sigma-Aldrich, St. Louis, Mo.) were prepared using physical mixing. Two loadings (high dosage and low dosage) were prepared. 10 grams of Na-CMC were slowly added into 290 gram of PBS solution with magnetic stirring at 60° C. to prepare 3 wt % Na-CMC solution. The pre-mixed solution was heated up to 121° C. for 20 minutes to make a homogenous gel solution. The high dosage gel composition (2 mg Ketorolac/3 g Na-CMC) was prepared by adding 24 mg of Ketorolac to 36 g of 3 wt % Na-CMC gel. The mixture was manually mixed with a spatula for 5 minutes. 3 g of gel were weighed into 5 ml syringes with a 3-way stopcock. Syringes were autoclaved for 20 minutes at 121° C. using an AMSCO Steam Powered Sterilizer, Model #3021, SN 0100593-08. The low dosage gel composition (0.3 mg Ketorolac/3 g Na-CMC) was prepared by adding 3.6 mg of Ketorolac to 36 g of 3 wt % Na-CMC gel. The mixture was manually mixed using a spatula for 5 minutes. 3 g of gel were weighed into 5 ml syringes with a 3 way stopcock. Syringes were autoclaved for 20 minutes at 121° C. using AMSCO Steam Powered Sterilizer, (Model #3021, SN 0100593-08).

### In Vivo Testing of the Effects of Ketorolac Gel and Naproxen Gel on POI

The charcoal transit model is an acceptable and established model to study the effects of abdominal surgery on intestinal motility. We followed the procedure as described by De Winter et al. (1997) (Reference: Effect of adrenergic and nitrergic blockade on experimental ileus in rats. British Journal of Pharmacology (1997) 120, 464-468. Benedicte Y. De Winter, Guy E. Boeckxstaens, Joris G. De Man, Tom G. Moreels, Arnold G. Herman & Paul A. Pelckmans). The model simulates the abdominal procedure in human and tests for gastric emptying and intestinal motility by the transfer of a charcoal meal fed to the animals.

### Methods of Treatment

### Surgeries

Male Sprague Dawley rats, weighing 250-275 g (8 in group) were anesthetized with a xylazine/ketamine cocktail, injected IM, the abdomen was shaved and disinfected with alcohol and Betadyne. A 2.5 cm incision was made from the xyphoid process caudally. The intestinal contents was exteriorized onto wet gauze. The cecum was manipulated gently for 1 minute. The abdominal contents were replaced, the treatment was applied, and the body wall was closed with 4-0 PDS for the fascia and muscle layers. The skin was closed with wound clips. In addition, two control groups were tested as well: incision only and naïve. Treatment groups included two control groups, surgery (incision only) and naïve, and 4 treatment groups, a high and low dose of Diclofenac, a high and a low dose of Naproxen, as well as 3 wt % Na-CMC gel alone. The high and low dose of Diclofenac, as well as the 3 wt % Na-CMC gel were prepared as described above. 8 rats were included in each group with a total of 56 rats.

### Charcoal Transit

The rats were treated with an oral solution of charcoal (10%) in acacia gum (2%). Four hours following oral administration rats were euthanized with Euthasole. The intestines were exteriorized and the distance traveled by the charcoal meal was measured as well as the full length of the intestines (from the pylorus through the anus). Results are expressed as percent of distance traveled relative to the full length of the intestines.

### Study Groups

### Results

| | Dose | % GI | |
|---|---|---|---|
| Study Group | (mg) | Transfer | SE |
| Naïve (no surgery) | NA | 83.38 | 2.51 |
| Control (surgery - no | NA | 53.92 | 2.88 |
| treatment) | | | |
| Ketorolac high | 2.0 | 69.84 | 3.65 |
| Ketorolac low | 0.3 | 70.99 | 4.62 |
| Naproxen high | 2.0 | 65.34 | 3.03 |
| Naproxen low | 0.3 | 56.9 | 2.5 |
| Gel only | NA | 52.04 | 4.29 |

It was observed that intestinal manipulation decreases gastric emptying and intestinal motility in the rat in the same manner that has been described in humans undergoing intestinal surgery. A dose response was not observed using the current Ketorolac low and high dose formulations, but a significant alleviation of the POI was observed in both doses. Similarly, when the naproxen was applied to the manipulated intestines, a dose related prevention of the POI was observed. Unlike the diclofenac, where the high dose completely alleviated the POI, the high dose of naproxen was not completely curative, yet, both doses improved on the gastric emptying and intestinal transfer compared with the non-treated animals and the animals treated with the gel alone. The gel alone had no significant effect on the POI.

Although this invention has been shown and described with respect to detailed embodiments thereof, it will be understood by those skilled in the art that various changes in form and detail thereof may be made without departing from the spirit and scope of the claimed invention.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. A method of preventing or reducing postoperative ileus and gastric stasis, which method comprises administering directly to serosal surfaces of gastrointestinal and other visceral organs a therapeutically effective amount of a composition which comprises a carrier component and a bioactive component.

17. The method according to claim 16 wherein the bioactive component comprises a nonsteroidal anti-inflammatory drug selected from the group consisting of propionic acid derivatives, acetic acid derivatives, fenamic acid derivatives, biphenylcarboxylic acid derivatives, alkali metal salts thereof, and combinations thereof.

18. The method of claim 16 wherein the composition is in a form selected from the group consisting of an injectable gel, a sprayable gel, an injectable liquid, and a sprayable liquid.

19. The method of claim 18 wherein said injectable gel, sprayable gel, injectable liquid, sprayable liquid comprises an aqueous solvent and a gelling material.

20. The method of claim 19 wherein said aqueous solvent is selected from the group consisting of physiological buffer solution, saline and water.

21. The method of claim 19 wherein said aqueous solvent solution is selected from the group consisting of buffered saline, hypertonic saline, phosphate buffer solution, hypertonic buffer, Hank's balanced salts solution, Tris buffered saline, Hepes buffered saline and combinations thereof.

22. The method of claim 19 wherein said gelling material is selected from the group consisting of salts of carboxymethyl cellulose, carboxymethyl cellulose, carboxyethyl cellulose, hyaluronic acid, salts of hyaluronic acid, alginate, cross-linked alginate and combinations thereof.

23. The method of claim 22 wherein said gelling material comprises salts of carboxymethyl cellulose wherein said salts of carboxymethyl cellulose is sodium carboxymethyl cellulose.

24. The method of claim 16 wherein said carrier component is an injectable gel comprising phosphate buffered saline and sodium carboxymethyl cellulose; and wherein said bioactive component is an nonsteroidal anti-inflammatory drug.

25. The method of claim 16 wherein said carrier component is an injectable gel comprising phosphate buffered saline and sodium carboxymethyl cellulose; and wherein said bioactive component is diclofenac.
